(19) United States
(12) Patent Application Publication
(10) Pub. No.: US 2008/0097336 A1
(43) Pub. Date: Apr. 24, 2008

(54) Needle protective device

(75) Inventors: Chien-Yuan Lee, Tamsui Town (TW); Chih-Cheng Kao, Taipei (TW)

Correspondence Address: BACON & THOMAS, PLLC, 625 SLATERS LANE, FOURTH FLOOR, ALEXANDRIA, VA, 22314, US

(73) Assignee: Linfair Engineering (HK) Co., Ltd., Chai Wan (HK)

(21) Appl. No.: 11/451,339

(22) Filed: Jun. 13, 2006

(51) Int. Cl. A61M 5/32

(52) U.S. Cl. 604/198

(57) ABSTRACT

A needle protective device for a syringe having a barrel has a needle hub and a sleeve. The needle hub detachably connected to the barrel has an anti-rotation device. The anti-rotation device is formed on the needle hub. The sleeve is mounted slidably on the barrel and has an inner wall, a detent and an anti-rotation seat. The detent is formed at the inner wall of the sleeve. The anti-rotation seat is formed inside the sleeve and selectively engages the anti-rotation device of the needle hub so that the needle can be attached to the barrel. After injecting liquid material, the barrel is pulled out of the sleeve, the needle hub enlarges size after being stopped by the detent in the sleeve so the barrel can be detached from the needle hub that is held in the sleeve. Consequently, detaching the barrel from the needle hub is easy.

### BACKGROUND OF THE INVENTION

1. Field of the Invention

The present invention relates to a syringe, and more particularly to a syringe with a needle protective device that is able to prevent a contaminated needle from inadvertently stabbing and infecting a person.

2. Description of Related Art

A conventional syringe in accordance with the prior art comprises a barrel, a needle hub and a sleeve.

The barrel is hollow, selectively holds a liquid material such as water, liquid medication or the like and has a rear opening, a front protrusion and a plunger. The rear opening is formed on the barrel. The front protrusion is formed longitudinally on the barrel and has an outer wall and an external thread. The external thread is formed on the outer wall of the front protrusion. The plunger is slidably mounted in the barrel, protrudes from the rear opening, draws liquid material into the barrel and ejects liquid material from the barrel into a subject such as a human, animal, an experimental material, industrial material or the like.

The needle hub is attached detachably to the front protrusion and has a front end, a rear end, a needle and an internal thread. The needle is used to draw material into the barrel and eject the material from the barrel into the subject, is mounted longitudinally through the needle hub and protrudes from the front end of the needle hub. The internal thread is formed in the rear end of the needle hub and selectively screws onto the external thread of the front protrusion on barrel to attach the needle hub to the barrel.

The sleeve is hollow and has a front opening, a rear opening, an inner wall and a detent. The front opening and the rear opening are formed on the sleeve and communicate with each other. The rear opening allows the barrel to extend into the sleeve so the needle on the needle hub is able to extend out of the front opening in the sleeve. The detent is formed inside of the inner wall of the sleeve and selectively engages the needle hub to disconnect the needle hub from the barrel when the needle hub and the barrel are unscrewed from each other. Since the needle is inside the sleeve, a contaminated needle will not inadvertently stab a person handling the used needle.

However, screwing and unscrewing the needle hub from the barrel waste a lot of time. Furthermore, the detent of the barrel has no stopper formed inside so the needle hub may rotate inside the detent of the barrel and the needle hub is unscrewed from the barrel. Disconnecting the needle hub from the barrel wastes time and may not be easy. The conventional syringe is not very convenient to use.

To overcome the shortcomings, the present invention provides a needle protective device to obviate or mitigate the aforementioned problems.

### SUMMARY OF THE INVENTION

The primary objective of the present invention is to provide a needle protective device to allow a needle hub to be disconnected easily from a barrel of a syringe.

The needle protective device in accordance with the present invention for a syringe having a barrel has a needle hub and a sleeve. The needle hub connected detachably to the barrel has an anti-rotation device. The anti-rotation device is formed on the needle hub. The sleeve is mounted slidably on the barrel and has an inner wall, a detent and an anti-rotation seat. The detent is formed in the inner wall of the sleeve. The anti-rotation seat is formed inside the sleeve and selectively engages the anti-rotation device of the needle hub so the needle hub can be attached to the barrel. After the liquid material has been injected into a subject, the barrel is pulled out of the sleeve, and the needle hub enlarges after stopping at the detent of the sleeve so the barrel can be detached from the needle hub. Disconnecting the needle hub from the barrel is easy.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a syringe with a first embodiment of a needle protective device for a syringe in accordance with the present invention;

FIG. 2 is an exploded perspective view of the needle protective device and the syringe in FIG. 1;

FIG. 3 is an operational side view in partial section of the syringe and the needle protective device in FIG. 1 when the barrel is pushed into the sleeve;

FIG. 4 is an operational side view in partial section of the syringe and the needle protective device in FIG. 1 when the needle hub is connected to the barrel;

FIG. 5 is an operational side view in partial section of the syringe and the needle protective device in FIG. 1 when the barrel is pulled part way out of the sleeve;

FIG. 6 is an operational side view in partial section of the syringe and the needle protective device in FIG. 1 when the barrel has been pulled completely out of the sleeve and the needle hub is disconnected from the barrel and held in the sleeve;

FIG. 7 is an operational front view in partial section of the needle protective device for a syringe in FIG. 1 before resilient spiral fingers on the needle hub engage recesses in the sleeve;

FIG. 8 is an operational front view in partial section of the needle protective device for a syringe in FIG. 1 when the resilient spiral fingers on the needle hub engage the recesses in the sleeve;

FIG. 9 is a perspective view of a syringe and a second embodiment of the needle protective device for a syringe in accordance with the present invention;

FIG. 10 is an exploded perspective view of the needle protective device and the syringe in FIG. 9;

FIG. 11 is an operational side view in partial section of the syringe and the needle protective device in FIG. 9 when the barrel is pushed into the sleeve;

FIG. 12 is an operational side view in partial section of the syringe and the needle protective device in FIG. 9 when the needle hub is connected to the barrel;

FIG. 13 is an operational side view in partial section of the syringe and the needle protective device in FIG. 9 when the barrel is pulled part way out of the sleeve;

FIG. 14 is an operational side view in partial section of the syringe and the needle protective device in FIG. 9 when the barrel has been pulled completely out of the sleeve and the needle hub is disconnected from the barrel and held in the sleeve;

FIG. 15 is an operational front view in partial section of the needle protective device for a syringe in FIG. 9 before stoppers on the needle hub engage longitudinal recesses in the sleeve;

FIG. 16 is an operational front view in partial section of the needle protective device for a syringe in FIG. 9 when stoppers on the needle hub engage the longitudinal recesses in the sleeve; and

FIG. 17 is an operational side view in partial section of another syringe and a third embodiment of the needle protective device for a syringe in accordance with the present invention when the barrel has been pulled completely out of the sleeve and the needle hub disconnected from the barrel and held in the sleeve.

### DETAILED DESCRIPTION OF THE PREFERRED EMBODIMENT

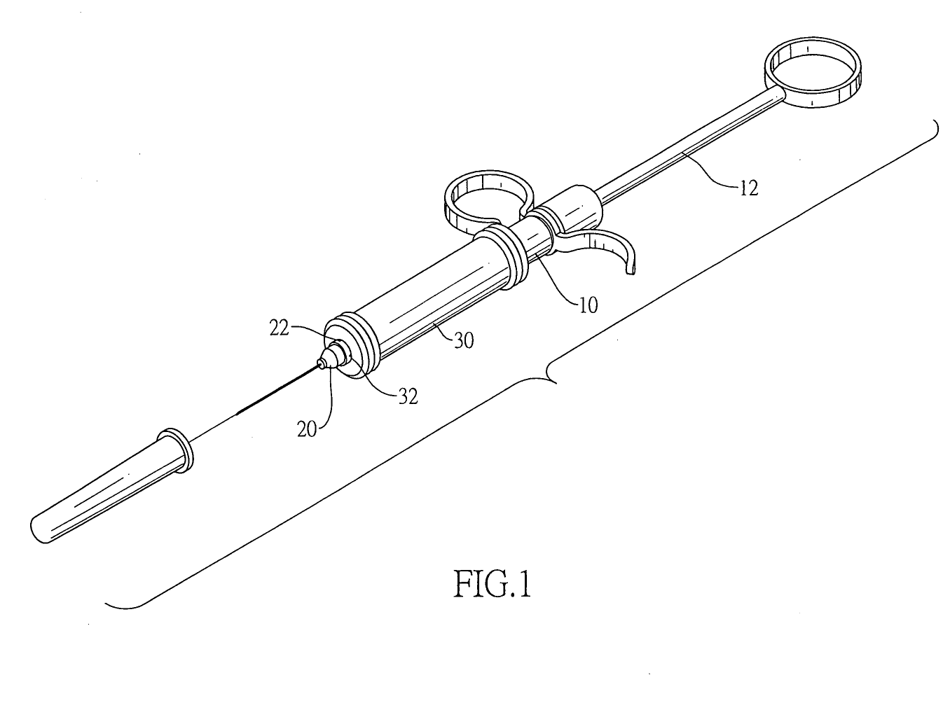

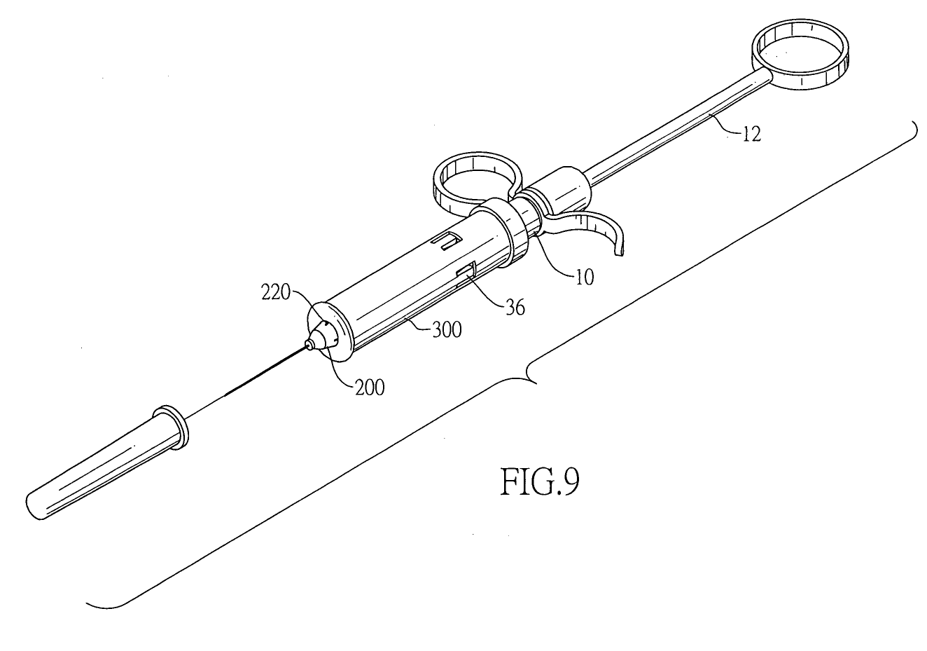

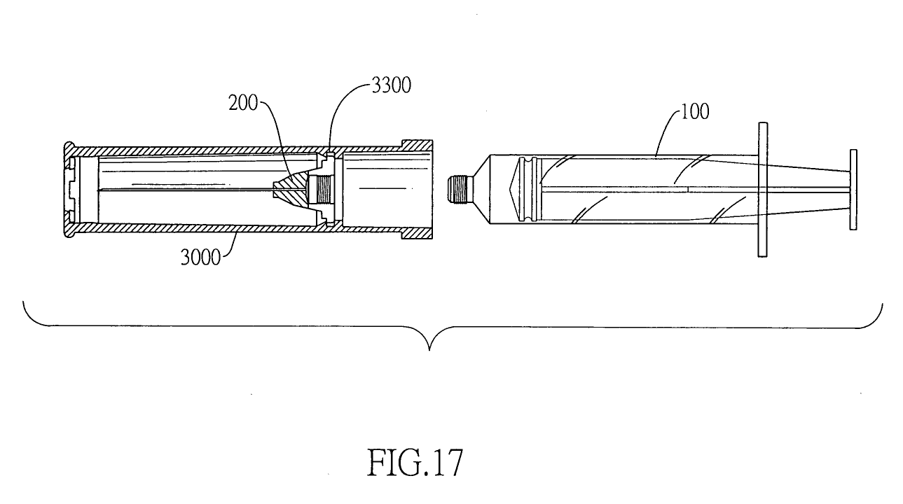

With reference to FIGS. 1, 9 and 17, a needle protective device in accordance with the present invention for a syringe having a barrel (10, 100) has a needle hub (20, 200) and a sleeve (30, 300, 3000).

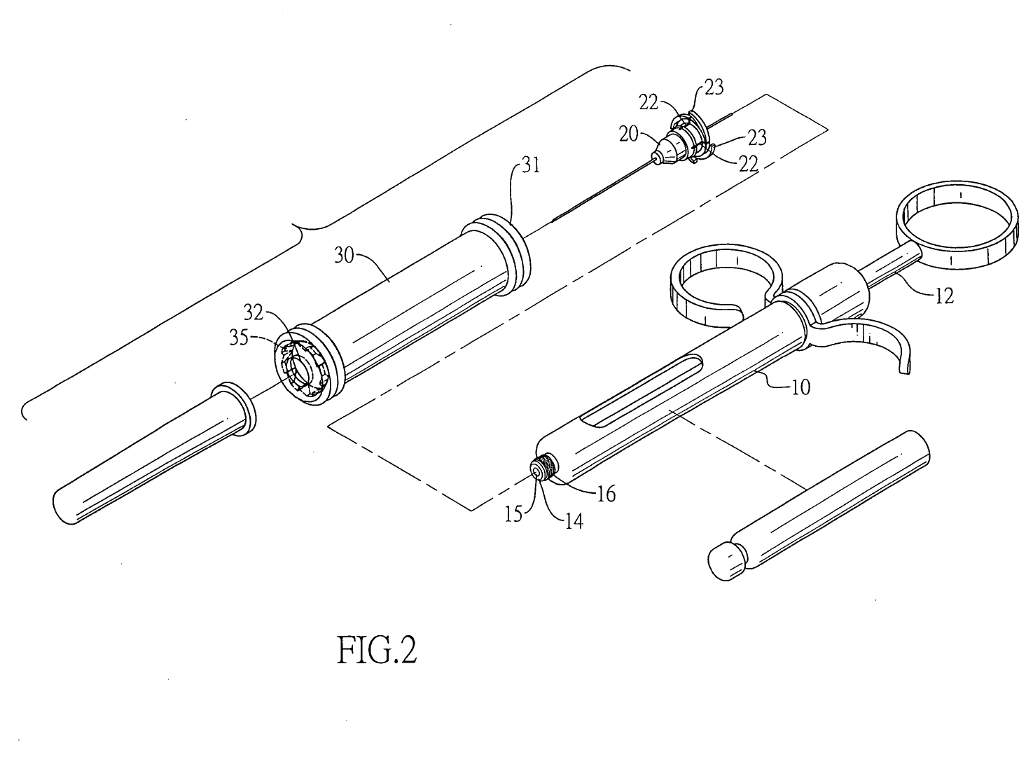

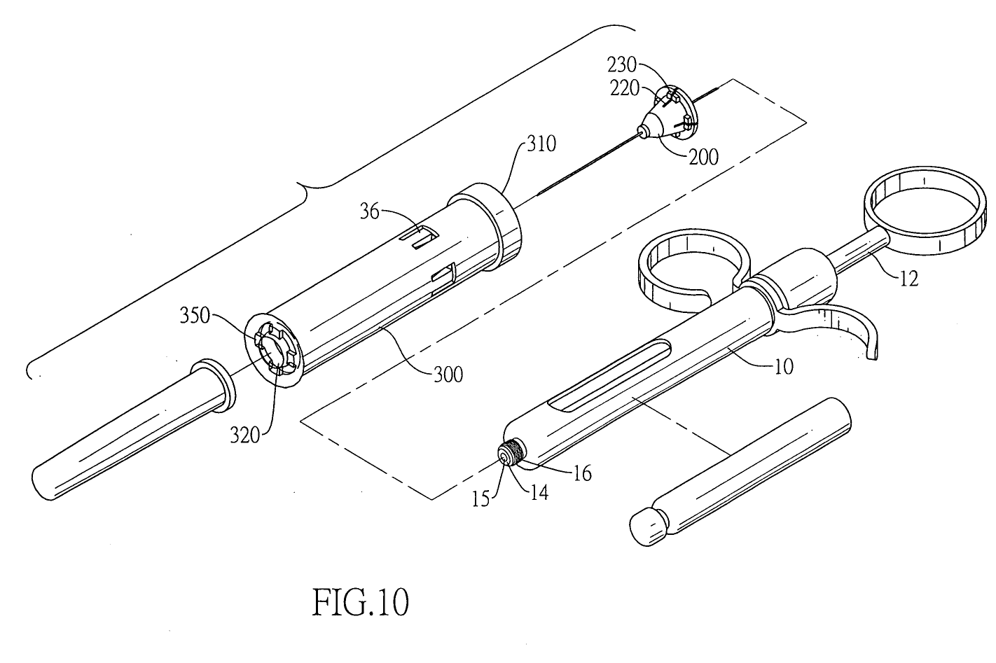

With further reference to FIGS. 2 and 10, the barrel (10, 100) is hollow, holds a liquid material such as water, liquid medication or the like and has a front end, a rear opening, a sidewall, an optional window, a plunger (12) and a protrusion (14). The rear opening is formed on the barrel (10, 100). The window is formed longitudinally in the sidewall of the barrel (10). The plunger (12) is mounted slidably in the barrel (10, 100), injects liquid material into a such as a human, animal, an experimental material, industrial material or the like, protrudes from the rear opening and may be used to draw liquid material into the barrel (10, 100), has a front end and may have a liquid material cartridge. The liquid material cartridge is prefilled with liquid material and is mounted on the front end of the plunger (12). The protrusion (14) is formed on and protrudes longitudinally from the front end of the barrel (10, 100) and has a central through hole (15), a sidewall and an external thread (16). The central through hole (15) is formed coaxially through the protrusion (14). The external thread (16) is formed on the sidewall of the protrusion (14).

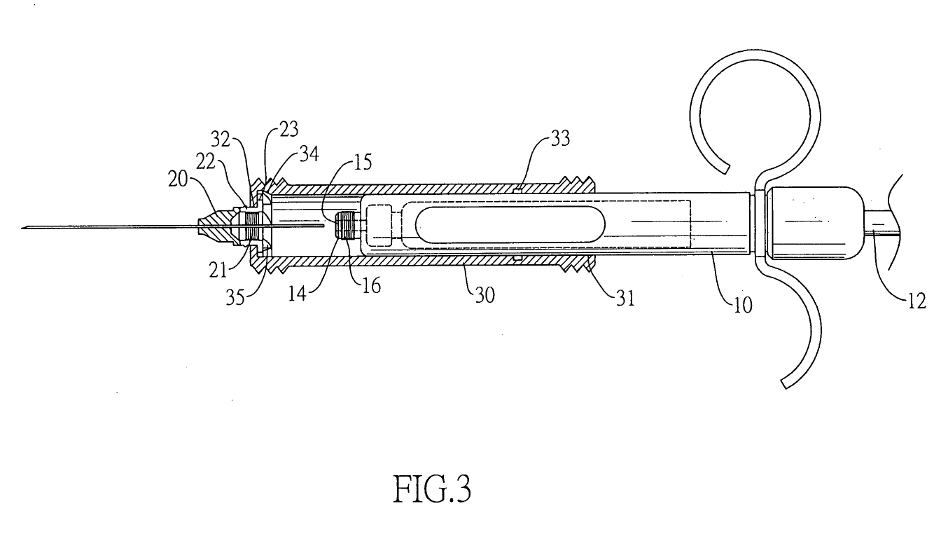

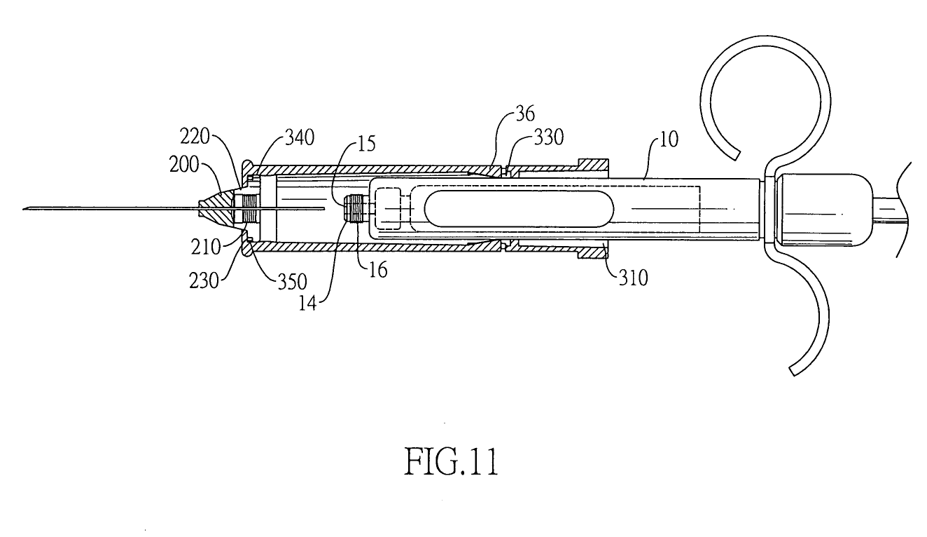

With further reference to FIGS. 3 and 11, the needle hub (20, 200) is resilient and has a needle, an inner end, a sidewall, an edge, an internal thread (21, 210), multiple optional expansion slots (22, 220) and an anti-rotation device.

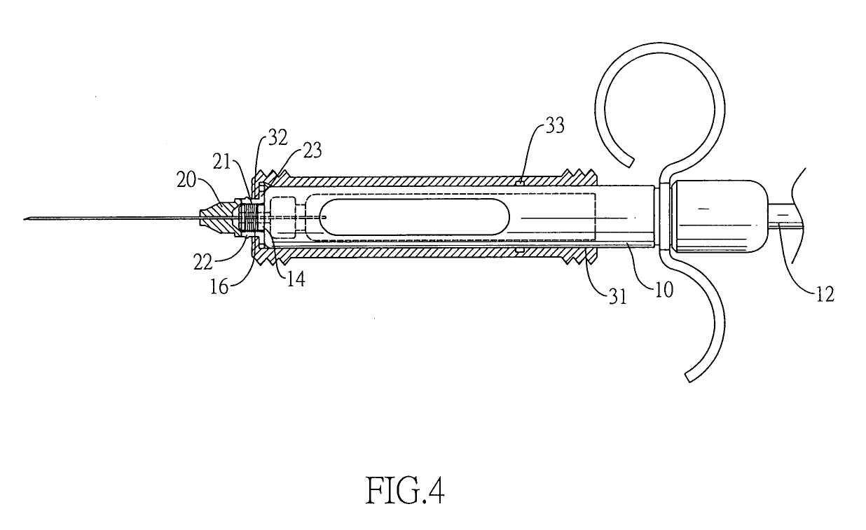

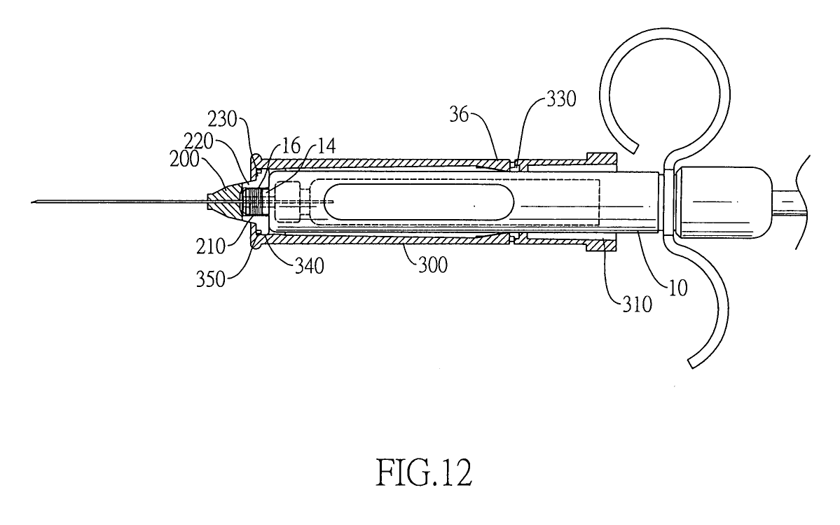

The needle is mounted coaxially through and protrudes from the needle hub (20, 200) and has an inner end and an outer end. With further reference to FIGS. 4 and 12, the inner end extends through the central through hole (15) in the protrusion (14) into the barrel (10, 100).

The edge is formed at the inner end.

The internal thread (21, 210) is formed coaxially in the inner end of the needle hub (20, 200) and selectively screws onto the external thread (16) on the protrusion (14) of the barrel (10, 100) to attach the needle hub (20, 200) to the barrel (10, 100).

The expansion slots (22, 220) allow the inner end of the needle hub (20, 200) to be expanded or contracted, are formed longitudinally through the sidewall of the needle hub (20, 200) and communicate with the inner end of the needle hub (20, 200).

The anti-rotation device is formed on the needle hub (20, 200), keeps the needle hub (20, 200) from rotating when the protrusion (14) on the barrel (10) is screwed into the needle hub (20, 200) and may be multiple resilient spiral pawls (23) or stoppers (230).

In a first embodiment of the needle protective device for a syringe in accordance with the present invention, the anti-rotation device is multiple resilient spiral pawls (23) are formed separately at and extending out from the edge of the needle hub (20).

In a second embodiment of the needle protective device for a syringe in accordance with the present invention, the anti-rotation device is multiple stoppers (230) are formed on and protruding radially out from the sidewall of the needle hub (200).

The sleeve (30, 300, 3000) is hollow, is mounted slidably on the barrel (10, 100), holds the needle hub (20, 200) and has an inner wall, a rear opening (31, 310), a front end, a front hole (32, 320), an annular detent (33, 330, 3300), multiple optional resilient clamps (36), an optional hub seat (34, 340) and an anti-rotation seat.

The rear opening (31, 310) is formed coaxially on the sleeve (30, 300, 3000) and allows the barrel (10, 100) to extend into the sleeve (30, 300, 3000).

The front hole (32, 320) is formed coaxially on the sleeve (30, 300, 3000), communicates with the rear opening (31, 310) in the sleeve (30, 300, 3000) and allows the needle and the needle hub (20, 200) to protrude longitudinally from the sleeve (30, 300, 3000).

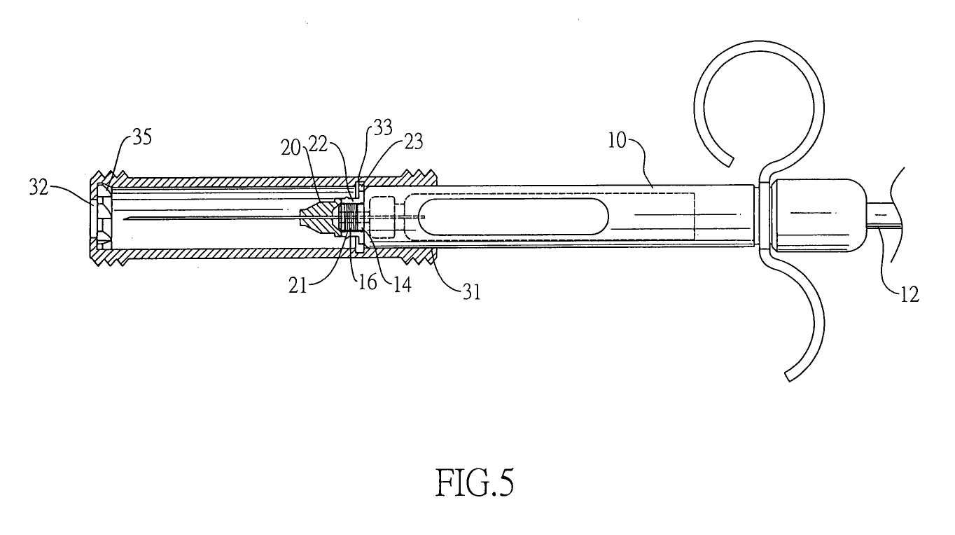

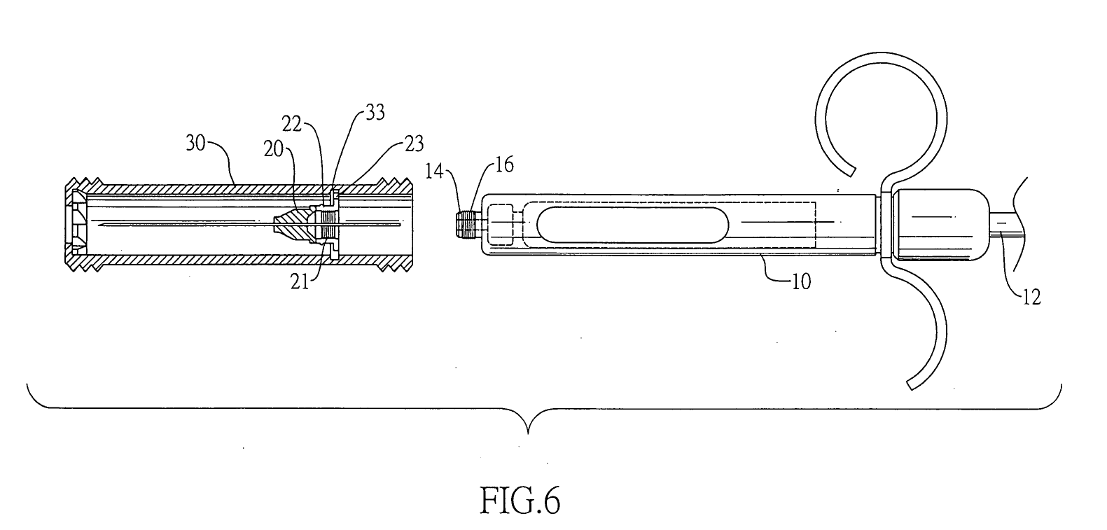

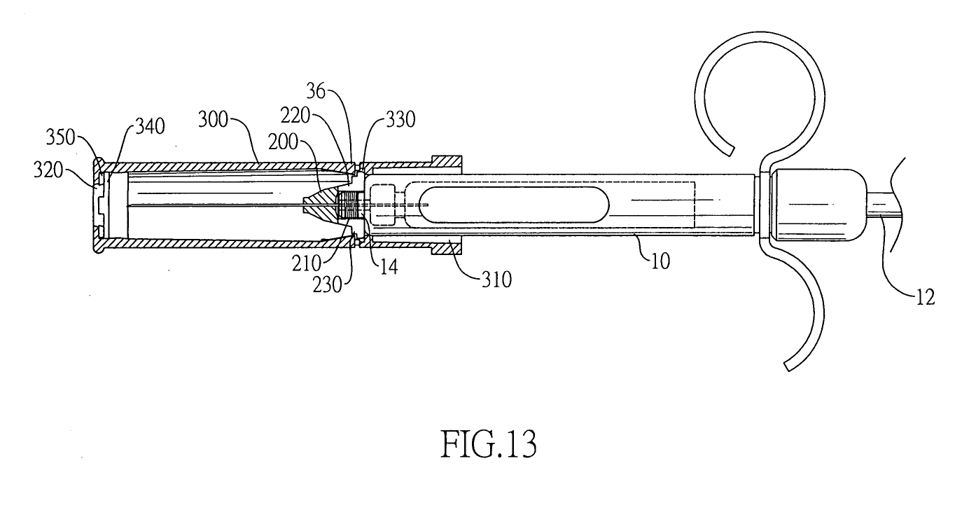

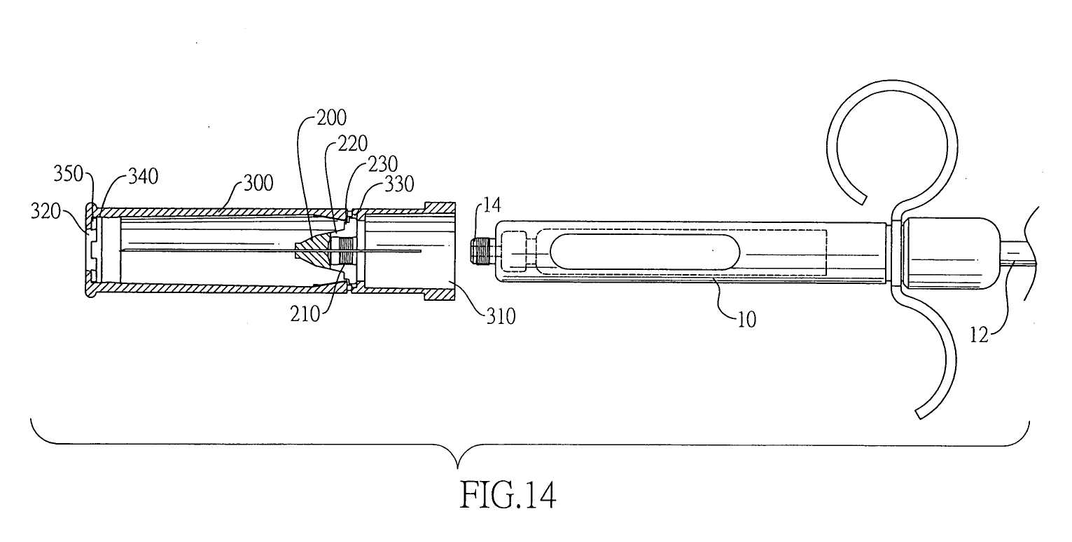

With further reference to FIGS. 5, 6, 13,14 and 17, the detent (33, 330, 3300) is formed in the inner wall of the sleeve (30, 300, 3000) near the rear opening (31, 310), selectively engages the edge of the needle hub (20, 200) and may be an annular groove or multiple through slots. In the first embodiment of the needle protective device for a syringe, the detent (33) is an annular groove. In the second embodiment of the needle protective device for a syringe, the detent (330) is multiple through slots. The detent (330) may also be formed between two annular detents. The annular detents are formed at inner wall of the sleeve (300). The through slots are formed around and through the inner wall of the sleeve (300). In the third embodiment of the needle protective device for a syringe, the detent (3300) may also be formed between two annular extensions. The annular extensions are formed at inner wall of the sleeve (3000).

In the second embodiment of the needle protective device for a syringe, the resilient clamps (36) are formed through the sleeve (300), correspond respectively to and communicate respectively with the through slots forming the detent (330), are pressed out by the edge of the needle hub (200) when the needle hub (200) is drawn into the sleeve (300) by the barrel (10) and clamp the needle hub (200) in position when the edge of the needle hub (200) aligns with the through slots forming the detent (330). With the needle hub (200) clamped in the detent (330), the needle and the needle hub (200) are held securely in the sleeve (300) when the barrel (10) is separated from the needle hub (200).

The hub seat (34, 340) is formed on the inner wall of the sleeve (30, 300, 3000) adjacent to the front hole (32, 320) of the sleeve (30, 300, 3000), holds the needle hub (20, 200) in position and is implemented as a detent. In the first embodiment of the needle protective device for a syringe, the detent is implemented as a shallow annular groove. The expansion slots (22) in the needle hub (20), allow the edge at the inner end of the needle hub (20) to expand so the needle hub (20) is held in the hub seat (34). In the second embodiment of the needle protective device for a syringe, the detent is formed by a slight constriction in the inner wall of the sleeve (300). The expansion slots (220) allow the edge at the inner end of the needle hub (200) to be compressed and pass over the constriction in the inner wall of the sleeve (300) and be held in the hub seat (340). Furthermore, when the needle hub (20, 200) is in the hub seat (34, 340), the front end of the sleeve (30, 300) expends to hold the needle hub (20, 200) at the position of the hub seat (34, 340).

The anti-rotation seat is formed inside the sleeve (30, 300, 3000) adjacent to the front hole (32, 320), selectively engages the anti-rotation device of the needle hub (20, 200) to keep the needle hub (20, 200) from rotating so the needle hub (20, 200) can be screwed into the barrel (10, 100) and may be multiple spiral recesses (35) or multiple longitudinal recesses (350).

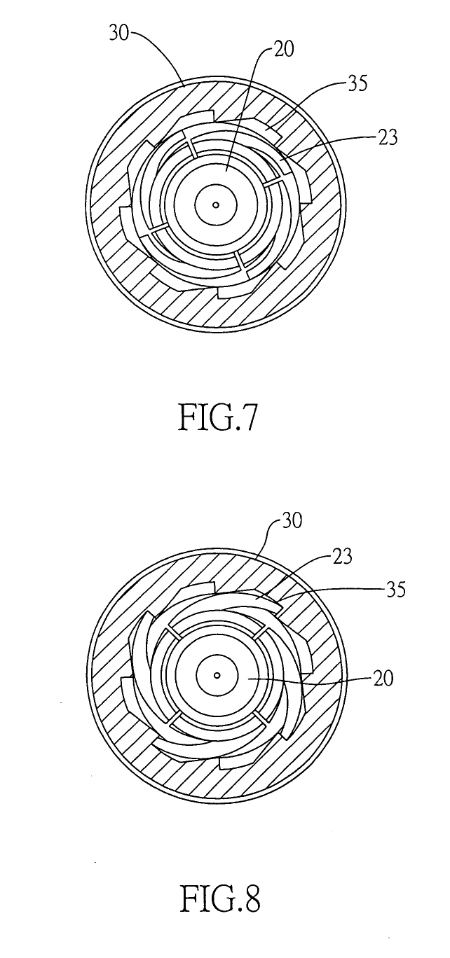

With further reference to FIGS. 7 and 8, the anti-rotation seat in the first embodiment of the needle protective device is multiple spiral recesses (35) formed inside the sleeve (30) adjacent to the front hole (32) of the sleeve (30), selectively engaging the resilient spiral pawls (23) of the needle hub (20) when the needle hub (20) is placed completely into the barrel (10) and keeping the needle hub (20) from rotating so the needle hub (20) can be screwed into the barrel (10, 100) and detaching the needle hub (20) from the barrel (10) is easy.

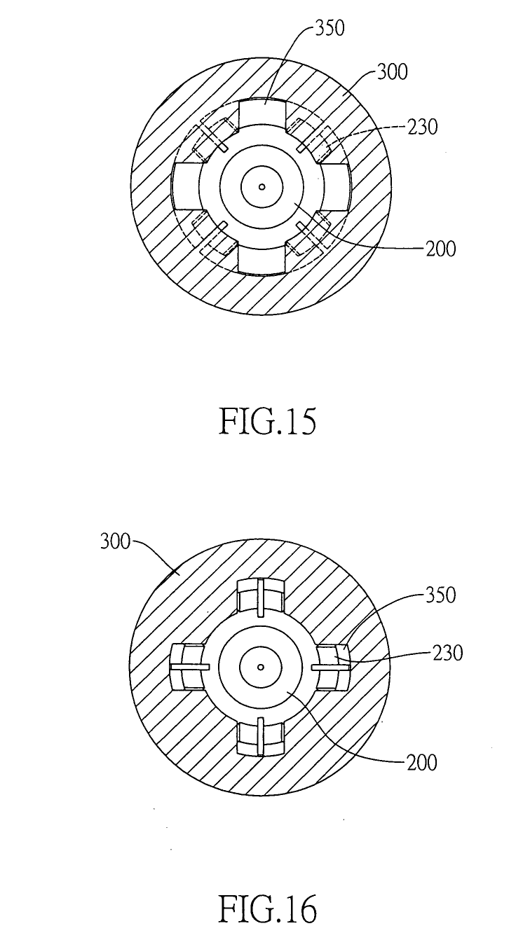

With further reference to FIGS. 15 and 16, the anti-rotation seat in the second embodiment of the needle protective device is multiple longitudinal recesses (350) formed inside the sleeve (300) adjacent to the front hole (320) of the sleeve (300), engaging the stoppers (230) of the needle hub (200) when the needle hub (200) is placed completely into the barrel (10) and keeping the needle hub (200) from rotating so the needle hub (200) can be screwed into the barrel (10, 100) and detaching the needle hub (200) from the barrel (10, 100) is easy.

Even though numerous characteristics and advantages of the present invention have been set forth in the foregoing description together with details of the structure and function of the invention, the disclosure is illustrative only. Changes may be made in detail especially in matters of shape, size, and arrangement of parts within the principles of the invention to the full extent indicated by the broad general meaning of the terms in which the appended claims are expressed.

What is claimed is:

1. A needle protective device for a syringe having a barrel having a front end and a protrusion formed on the front end of the barrel and having an external thread and a central through hole formed coaxially through the protrusion, the needle protective device comprising
a needle hub having
a needle being mounted coaxially through and protruding from the needle hub and having
an inner end; and
an outer end;
an inner end;
a sidewall;
an edge formed at the inner end;
an internal thread being formed coaxially in the inner end of the needle hub; and
an anti-rotation device being formed on the needle hub to keep the needle hub from rotating when the needle hub is screwed into the protrusion on the barrel; and
a sleeve being hollow holding the needle hub and having
an inner wall;
a rear opening being formed coaxially on the sleeve;
a front hole being formed coaxially on the sleeve and communicating with the rear opening in the sleeve to allow the needle hub to protrude longitudinally from the sleeve;
a detent being formed in the inner wall of the sleeve near the rear opening and selectively engaging the edge of the needle hub; and
an anti-rotation seat being formed on the inner wall of the sleeve adjacent to the front hole and selectively engaging the anti-rotation device of the needle hub.

2. The needle protective device for a syringe as claimed in claim 1, wherein the needle hub further has multiple expansion slots being formed longitudinally through the sidewall of the needle hub and communicating with the inner end of the needle hub.

3. The needle protective device for a syringe as claimed in claim 1, wherein
the anti-rotation device comprises multiple resilient spiral pawls formed separately at and extending out from the edge of the needle hub;
the anti-rotation seat comprises multiple spiral recesses formed inside the sleeve adjacent to the front hole of the sleeve and selectively engaging the resilient spiral pawls of the needle hub.

4. The needle protective device for a syringe as claimed in claim 1, wherein
the anti-rotation device comprises multiple stoppers formed on and protruding out from the sidewall of the needle hub; and
the anti-rotation seat comprises multiple longitudinal recesses formed inside the sleeve adjacent to the front hole of the sleeve and engaging the stoppers of the needle hub.

5. The needle protective device for a syringe as claimed in claim 1, wherein the detent is formed between two annular extensions in the inner wall of the sleeve.

6. The needle protective device for a syringe as claimed in claim 1, wherein the detent is an annular groove.

7. The needle protective device for a syringe as claimed in claim 1, wherein
the detent comprises multiple through slots formed around and through the inner wall of the sleeve;
the sleeve further has multiple resilient clamps formed through the sleeve, corresponding respectively to and communicating respectively with the through slots forming the detent, being pressed out by the edge of the needle hub when the needle hub is drawn into the sleeve by the barrel and clamping the needle hub in position when the edge of the needle hub aligns with the through slots forming the detent.

8. The needle protective device for a syringe as claimed in claim 1, wherein the sleeve further has a hub seat formed on the inner wall of the sleeve adjacent to the front hole of the sleeve to hold the needle hub in position and implemented as a detent.

9. The needle protective device for a syringe as claimed in claim 8, wherein the detent is an annular groove.

10. The needle protective device for a syringe as claimed in claim 8, wherein the detent is formed by a slight constriction in the inner wall of the sleeve.
